(19) United States
(12) Patent Application Publication
(10) Pub. No.: US 2013/0244270 A1
(43) Pub. Date: Sep. 19, 2013

(54) MICROFLUIDIC DEVICE FOR CELL MOTILITY SCREENING AND CHEMOTAXIS TESTING

(71) Applicants: Lan Xie, Beijing (CN); Rui Ma, Beijing (CN); Chao Han, Beijing (CN); Wanli Xing, Beijing (CN); Jing Cheng, Beijing (CN)

(72) Inventors: Lan Xie, Beijing (CN); Rui Ma, Beijing (CN); Chao Han, Beijing (CN); Wanli Xing, Beijing (CN); Jing Cheng, Beijing (CN)

(73) Assignees: Tsinghua University, Beijing (CN); CapitalBio Corporation, Beijing (CN)

(21) Appl. No.: 13/814,426

(22) PCT Filed: Aug. 10, 2011
(86) PCT No.: PCT/CN11/01329

§ 371 Date: May 21, 2013

(30) Foreign Application Priority Data
Aug. 10, 2010 (CN) 201010252513.3

(51) Int. Cl. C12Q 1/02

(52) U.S. Cl. CPC C12Q 1/025
USPC 435/29; 435/288.7

(57) ABSTRACT

The present invention relates to a microfluidic device used for cell motility screening and chemotaxis testing which comprises microfluidic channels and chambers. Cells which can secret a chemoattractant or chemorepellent are selectively planted in the microfluidic device and a stable chemoattractant or chemorepellent gradient can be established in the channels.

### TECHNICAL FIELD

The present disclosure relates to a microfluidic device and its uses for cell motility screening and chemotaxis testing.

### BACKGROUND ART

Microfluidic technology refers to a reaction system which could handle a small amount of liquid or samples (10⁻⁹-10⁻¹⁸ L) in microchannels in the scale of tens to hundreds of microns (Whitesides, Nature (2006) 442:368-73). The application of microfluidic technology in biochemical analysis originated from the research of capillary electrophoresis. Microfluidic technology has many desirable characteristics: ability of handling extremely small amount of samples; high sensitivity of separation and detection; low cost and low power consumption; high reaction speed; high integration, etc. These characteristics ensured that experiments could be performed in a continuous and efficient way. Up to now, microfluidic technology has been applied to research and analysis at the levels of molecules (e.g., DNA, protein, etc.), cells and tissues.

Motility is an important functional parameter for certain cells. For example, sperm motility is an important factor related to fertility. Currently, the swim-up method and the density-gradient centrifugal method are used clinically for sperm motility screening. However, these two methods may cause damage to sperms (such as oxygen free radicals explosion and DNA fragmentation) and thus affect its functions. Chemotaxis is the phenomenon in which eukaryotic cells, bacteria and other single-cell or multicellular organisms direct their movements according to certain chemicals in their environment. This is important for prokaryotic organisms to find nutrients and/or to avoid poisons. Chemotaxis is also critical for eukaryotic organisms, e.g., for sperm to find eggs during fertilization, for neurons or lymphocytes to migrate for their normal functions. Sperm chemotaxis refers to the movement along a chemoattractant concentration gradient and is an important mechanism for sperm to find eggs in vivo. It is one of the most important parameters related to sperm fertility. Chemotaxis assay uses a wide range of techniques available to evaluate the chemotactic activity of prokaryotic or eukaryotic cells. The most commonly used chemotaxis assays include the agar-plate technique, two-chamber technique and micro-video-recording technique. A basic requirement for a good chemotaxis assay is an effective and stable concentration gradient.

In 1995, a microfluidic chip was disclosed for sperm motility screening by Kricka & Wilding (U.S. Pat. No. 5,427,946). A cascade of branching microchannels was included between a sperm inlet pool and an oocyte positioning pool. This device facilitated the evaluation of sperm morphology and motility but sperm chemotaxis testing was not disclosed. Microfluidics was not used for sperm chemotaxis testing until 2003 (Koyama, Anal. Chem. (2006) 78:3354-9). The device by Koyama has three input channels and three output channels, connected by a chemotaxis chamber. Mouse sperms were introduced into the chemotaxis chamber between continuous flows of mouse ovary extract and blank buffer. The sperm experiencing chemotaxis swam toward the mouse ovary extract and was counted relative to those that swam toward the buffer. The disadvantage of this device lies in that it highly depends on the fluid stability and the shear force caused by the fluid manipulation is difficult to avoid.

### SUMMARY OF THE INVENTION

The present invention relates to a microfluidic device and its use for cell motility screening and chemotaxis testing. Therefore, in one aspect, provided herein is a microfluidic device for cell motility screening and/or chemotaxis testing, which comprises at least one motility screening channel, a buffering chamber and at least two branching channels, wherein the motility screening channel and the branching channels are connected to the buffering chamber.

In some embodiments, the branching channels may be symmetrically distributed around the buffering chamber. In some embodiments, the microfluidic device may further comprise an inlet pool and at least two outlet pools. In some embodiments, the inlet pool may be connected to the motility screening channel and the outlet pools may be connected to the branching channels. In some embodiments, the microfluidic device may comprise a top layer and a bottom layer, wherein the bottom layer is connected to the top layer. In some embodiments, the top layer may comprise the inlet pool and the outlet pool. In some embodiments, the bottom layer may comprise the motility screening channel, the buffering chamber and the branching channels. In some embodiments, the motility screening channel, the buffering chamber and/or the branching channels may be formed between the top layer and the bottom layer. In some embodiments, the top layer and/or bottom layer comprises or may be made of glass or PDMS. In some embodiments, the top layer and/or bottom layer may be about 2-10 mm thick. In some embodiments, the depth of the motility screening channel, the buffering chamber and/or the branching channels may be about 10-500 μm; the motility screening channel may be about 2-100 mm in length and about 50 μm-2 mm in width; and the branching channels may be about 2-100 mm in length and 50 μm-2 mm in width. In some embodiments, the diameter of the buffering chamber may be about 2-5 mm; and the diameter of the inlet pool and/or the outlet pools may be about 2-5 mm.

Further provided herein is a microfluidic system for cell motility screening and/or chemotaxis testing comprising a microfluidic device, which comprises at least one motility screening channel, a buffering chamber and at least two branching channels, wherein the motility screening channel and the branching channels are connected to the buffering chamber, and a chemoattractant, a chemorepellent, or a cell. In some embodiments, the microfluidic system may further comprise a liquid, which may be a buffer. In some embodiments, the chemoattractant or chemorepellent may form a gradient along the length of one of the branching channels. In some embodiments, the cell may be in one of the outlet pools, wherein the cell may be a cumulus cell. In some embodiments, the cumulus cell may come from a human or a mouse. In some embodiments, the cell may secret a chemoattractant or chemorepellent.

In another aspect, the present invention provides a method for cell motility screening and/or chemotaxis testing using a microfluidic device disclosed herein, comprising: a) adding the microfluidic device with a cell culture medium; b) adding a chemoattractant in one of the outlet pools; c) adding cells subject to the cell motility screening and/or chemotaxis testing to the inlet pool; and d) performing the cell motility screening and/or chemotaxis testing. Further provided herein is a method for cell motility screening and/or chemotaxis testing using a microfluidic device disclosed herein, comprising: a) adding the microfluidic device with a cell culture medium; b) adding a chemorepellent in one of the outlet pools; c) adding cells subject to the cell motility screening and/or chemotaxis testing to the outlet pools; and d) performing the cell motility screening and/or chemotaxis testing.

In some embodiments, the method may further comprise laying an oil, preferably mineral oil, on top of the microfluidic device. In some embodiments, the confluency of the cells subject to the cell motility screening and/or chemotaxis testing may be about 25-100%. In some embodiments, the method may further comprise refreshing the cell culture medium. In some embodiments, the chemoattractant and/or chemorepellent may be secreted by a cumulus cell. In some embodiments, the cells subject to the cell motility screening and/or chemotaxis testing may be sperms. In some embodiments, more than one chemoattractants and/or chemorepellents may be added to the outlet pools, wherein each outlet pool may comprise one chemoattractant and/or chemorepellent.

In some embodiments, the cell motility screening and/or chemotaxis testing may comprise comparing the number of cells moving towards and/or in the branching channels and/or the outlet pools. In some embodiments, the cell motility screening and/or chemotaxis testing may comprise calculating a chemotaxis index (CI), which is the ratio of the number of cells moving towards and/or in the branching channel and/or the outlet pools with the chemoattractant vs. the number of cells moving towards and/or in the branching channel and/or the outlet pools without the chemoattractant, or the ratio of the number of cells moving towards and/or in the branching channel and/or the outlet pools without the chemorepellent vs. the number of cells moving towards and/or in the branching channel and/or the outlet pools with the chemorepellent. In some embodiments, the number of cells is counted at a time point or multiple time points after adding cells subject to the cell motility screening and/or chemotaxis testing to the inlet pool. In some embodiments, the number of cells is counted by video recording. In some embodiments, at least 10, 100, 1000, 10,000 or more cells subject to the cell motility screening and/or chemotaxis testing are added to the inlet pool. In some embodiments, the method may further comprise collecting the cells in the branching channels and/or the outlet pools after the cell motility screening and/or chemotaxis testing.

### BRIEF DESCRIPTION OF THE DRAWINGS

FIG. 1 shows a three-dimensional view of an exemplary microfluidic device.

FIG. 2 shows a schematic view of an exemplary microfluidic device.

FIG. 3 shows a schematic view of an exemplary microfluidic device containing multiple motility screening channels.

FIG. 4 shows a schematic view of an exemplary microfluidic device containing multiple straight branching channels.

FIG. 5 is an illustration of the chemoattractant gradient formation.

FIG. 6 shows the counting area of an exemplary microfluidic device.

### DETAILED DESCRIPTION OF THE INVENTION

This invention provides a microfluidic device and its uses for cell motility screening and chemotaxis testing.

### A. DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a”, “an”, and “the” include plural references unless indicated otherwise. For example, “a” dimer includes one or more dimers.

As used herein, the term “microfluidic device” generally refers to a device through which materials, particularly fluid borne materials, such as liquids, can be transported, in some embodiments on a micro-scale, and in some embodiments on a nanoscale. Thus, the microfluidic devices described by the presently disclosed subject matter can comprise microscale features, nanoscale features, and combinations thereof.

Accordingly, an exemplary microfluidic device typically comprises structural or functional features dimensioned on the order of a millimeter-scale or less, which are capable of manipulating a fluid at a flow rate on the order of a μL/min or less. Typically, such features include, but are not limited to channels, fluid reservoirs, reaction chambers, mixing chambers, and separation regions. In some examples, the channels include at least one cross-sectional dimension that is in a range of from about 0.1 μm to about 500 μm. The use of dimensions on this order allows the incorporation of a greater number of channels in a smaller area, and utilizes smaller volumes of fluids.

A microfluidic device can exist alone or can be a part of a microfluidic system which, for example and without limitation, can include: pumps for introducing fluids, e.g., samples, reagents, buffers and the like, into the system and/or through the system; detection equipment or systems; data storage systems; and control systems for controlling fluid transport and/or direction within the device, monitoring and controlling environmental conditions to which fluids in the device are subjected, e.g., temperature, current, and the like.

As used herein, the terms “channel,” “micro-channel,” “fluidic channel,” and “microfluidic channel” are used interchangeably and can mean a recess or cavity formed in a material by imparting a pattern from a patterned substrate into a material or by any suitable material removing technique, or can mean a recess or cavity in combination with any suitable fluid-conducting structure mounted in the recess or cavity, such as a tube, capillary, or the like.

As used herein, the terms “flow channel” and “control channel” are used interchangeably and can mean a channel in a microfluidic device in which a material, such as a fluid, e.g., a gas or a liquid, can flow through. More particularly, the term “flow channel” refers to a channel in which a material of interest, e.g., a solvent or a chemical reagent, can flow through. Further, the term “control channel” refers to a flow channel in which a material, such as a fluid, e.g., a gas or a liquid, can flow through in such a way to actuate a valve or pump.

As used herein, “chip” refers to a solid substrate with a plurality of one-, two- or three-dimensional micro structures or micro-scale structures on which certain processes, such as physical, chemical, biological, biophysical or biochemical processes, etc., can be carried out. The micro structures or micro-scale structures such as, channels and wells, electrode elements, electromagnetic elements, are incorporated into, fabricated on or otherwise attached to the substrate for facilitating physical, biophysical, biological, biochemical, chemical reactions or processes on the chip. The chip may be thin in one dimension and may have various shapes in other dimensions, for example, a rectangle, a circle, an ellipse, or other irregular shapes. The size of the major surface of chips of the present invention can vary considerably, e.g., from about 1 mm² to about 0.25 m². Preferably, the size of the chips is from about 4 mm² to about 25 cm² with a characteristic dimension from about 1 mm to about 5 cm. The chip surfaces may be flat, or not flat. The chips with non-flat surfaces may include channels or wells fabricated on the surfaces.

The terms “chemoattractants” and “chemorepellents” refer to inorganic or organic substances possessing chemotaxis-inducer effect in motile cells. Effects of chemoattractants are elicited via chemotaxis receptors, and the chemoattractant moiety of a ligand is target cell specific and concentration dependent. Most frequently investigated chemoattractants are formyl peptides and chemokines. Responses to chemorepellents result in axial swimming and they are considered a basic motile phenomenon in bacteria. The most frequently investigated chemorepellents are inorganic salts, amino acids and some chemokines.

It is understood that aspects and embodiments of the invention described herein include “consisting” and/or “consisting essentially of” aspects and embodiments.

Other objects, advantages and features of the present invention will become apparent from the following specification taken in conjunction with the accompanying drawings.

### B. MICROFLUIDIC DEVICE AND MICROFLUIDIC SYSTEM

In one aspect, provided herein is a microfluidic device for cell motility screening and/or chemotaxis testing, which comprises at least one motility screening channel, a buffering chamber and at least two branching channels, wherein the motility screening channel and the branching channels are connected to the buffering chamber.

Any suitable number of branching channels and/or motility screening channels may be included in the microfluidic device. Typically, at least 2, 3, 4, 5, 10, 20, 50, 100 or more branching channels and/or motility screening channels may be included. In some embodiments, the branching channels and/or motility screening channels may be symmetrically distributed around the buffering chamber. Typically, the microfluidic device may further comprise the same number of outlet and inlet pools corresponding to the branching and motility screening channels, respectively. In some embodiments, the microfluidic device may further comprise an inlet pool and at least two outlet pools. In some embodiments, the inlet pool may be connected to the motility screening channel and the outlet pools may be connected to the branching channels. In some embodiments, the microfluidic device may comprise a top layer and a bottom layer, wherein the bottom layer is connected to the top layer. In some embodiments, the top layer may comprise the inlet pool and the outlet pool. In some embodiments, the bottom layer may comprise the motility screening channel, the buffering chamber and the branching channels. In some embodiments, the motility screening channel, the buffering chamber and/or the branching channels may be formed between the top layer and the bottom layer. In some embodiments, the top layer and/or bottom layer comprises or may be made of glass or PDMS. In some embodiments, the top layer and/or bottom layer may be about 2-10 mm thick. In some embodiments, the depth of the motility screening channel, the buffering chamber and/or the branching channels may be about 10-500 μm; the motility screening channel may be about 2-100 mm in length and about 50 μm-2 mm in width; and the branching channels may be about 2-100 mm in length and 50 μm-2 mm in width. In some embodiments, the diameter of the buffering chamber may be about 2-5 mm; and the diameter of the inlet pool and/or the outlet pools may be about 2-5 mm. In some embodiments, the microfluidic device for cell motility screening and/or chemotaxis testing may not have a motility screening channel, and may comprise only branching channels distributed symmetrically around the buffering chamber.

Further provide herein is a microfluidic system for cell motility screening and/or chemotaxis testing comprising a microfluidic device, which comprises at least one motility screening channel, a buffering chamber and at least two branching channels, wherein the motility screening channel and the branching channels are connected to the buffering chamber, and a chemoattractant, a chemorepellent, or a cell. In some embodiments, the microfluidic system may further comprise a liquid, which may be a buffer. In some embodiments, the chemoattractant or chemorepellent may form a gradient along the length of one of the branching channels. In some embodiments, the cell may be in one of the outlet pools, wherein the cell may be a cumulus cell. In some embodiments, the cumulus cell may come from a human or a mouse. In some embodiments, the cell may secret a chemoattractant or chemorepellent.

Other suitable exemplary microfluidic systems for cell motility screening and/or chemotaxis testing may also be provided. For example, an exemplary microfluidic system may comprise both a chemoattractant and a chemorepellent. The chemoattractant or chemorepellent may be added in the outlet pool or the inlet pool, and both may be added in a single outlet pool or inlet pool. The cells for cell motility screening and/or chemotaxis testing may be added in the inlet pool, or in the outlet pool. More than one chemoattractants and/or chemorepellent may be added to an exemplary microfluidic system, and each chemoattractant and/or chemorepellent may form a gradient along the length of one of the branching channels.

Exemplary microfluidic devices may comprise a central body structure in which various microfluidic elements are disposed. The body structure includes an exterior portion or surface, as well as an interior portion which defines the various microscale channels and/or chambers of the overall microfluidic device. For example, the body structure of an exemplary microfluidic devices typically employs a solid or semi-solid substrate that may be planar in structure, i.e., substantially flat or having at least one flat surface. Suitable substrates may be fabricated from any one of a variety of materials, or combinations of materials. Often, the planar substrates are manufactured using solid substrates common in the fields of microfabrication, e.g., silica-based substrates, such as glass, quartz, silicon or polysilicon, as well as other known substrates, i.e., gallium arsenide. In the case of these substrates, common microfabrication techniques, such as photolithographic techniques, wet chemical etching, micromachining, i.e., drilling, milling and the like, may be readily applied in the fabrication of microfluidic devices and substrates. Alternatively, polymeric substrate materials may be used to fabricate the devices of the present invention, including, e.g., polydimethylsiloxanes (PDMS), polymethylmethacrylate (PMMA), polyurethane, polyvinylchloride (PVC), polystyrene, polysulfone, polycarbonate and the like. In the case of such polymeric materials, injection molding or embossing methods may be used to form the substrates having the channel and reservoir geometries as described herein. In such cases, original molds may be fabricated using any of the above described materials and methods.

The channels and chambers of an exemplary device are typically fabricated into one surface of a planar substrate, as grooves, wells or depressions in that surface. A second planar substrate, typically prepared from the same or similar material, is overlaid and bound to the first, thereby defining and sealing the channels and/or chambers of the device. Together, the upper surface of the first substrate, and the lower mated surface of the upper substrate, define the interior portion of the device, i.e., defining the channels and chambers of the device. In some embodiments, the upper layer may be reversibly bound to the lower layer.

Exemplary systems may also include sample sources that are external to the body of the device per se, but still in fluid communication with the sample loading channel. In some embodiments, the system may further comprise an inlet and/or an outlet to the micro-channel. In some embodiments, the system may further comprise a delivering means to introduce a sample to the micro-channel. In some embodiments, the system may further comprise an injecting means to introduce a liquid into the micro-channel. Any liquid manipulating equipments, such as pipettes, pumps, etc., may be used as an injecting means to introduce a liquid to the micro-channel.

Advantages of an exemplary microfluidic device disclosed herein include:

1) Since the chemoattractant and/or chemorepellent is secreted by the cells planted in the outlet pools, a stable chemoattractant and/or chemorepellent gradient can be established in the buffering chamber as well as the straight branching channels. This is superior to other chemotaxis assays for which a stable fluid is difficult to maintain.

2) The in situ cultured cells can mimic the in vivo conditions well.

3) The straight branching channels are distributed around the buffering chamber symmetrically and different chemicals can be added in different outlet pools. The chemotaxis is tested among different outlet pools and the symmetry ensures or enhances the unbiasedness and effectiveness of the device.

4) Screening is achieved through the inherent motility of samples in a stable environment. Centrifugation is avoided which might cause potential damages to samples.

5) The device is easy to use, time-saving and labor-saving; the miniaturization of the device reduces the consumption of reagent and samples and is especially suitable for rare samples.

6) The top layer and bottom layer can be made up of PDMS which is quite permeable. PDMS can prevent or reduce the evaporation of water while is permeable for carbon dioxide and thus maintains a balanced system. Moreover, the top layer and bottom layer made of PDMS can be bonded together closely.

7) The microchannel can be sterilized and sealed by mineral oil and thus can avoid or reduce the pollution and reduce the damages.

8) The number and size of the motility screening channels and branching channels are quite flexible, in accordance with experimental requirement.

9) The device can be integrated with other microfluidic devices if necessary.

10) The fabrication of the microfluidic device is simple and materials of the device are cost-saving and reusable, which is easy to promote in ordinary laboratories.

### C. METHODS FOR CELL MOTILITY SCREENING AND/OR CHEMOTAXIS TESTING

In another aspect, the present invention provides a method for cell motility screening and/or chemotaxis testing using a microfluidic device disclosed herein, comprising: a) adding to the microfluidic device a cell culture medium; b) adding a chemoattractant in one of the outlet pools; c) adding cells subject to the cell motility screening and/or chemotaxis testing to the inlet pool; and d) performing the cell motility screening and/or chemotaxis testing. Further provided herein is a method for cell motility screening and/or chemotaxis testing using a microfluidic device disclosed herein, comprising: a) adding the microfluidic device with a cell culture medium; b) adding a chemorepellent in one of the outlet pools; c) adding cells subject to the cell motility screening and/or chemotaxis testing to the outlet pools; and d) performing the cell motility screening and/or chemotaxis testing.

Any suitable chemoattractants and/or chemorepellents may be added to the outlet pools for the cell motility screening and/or chemotaxis testing. In some embodiments, both a chemoattractant and a chemorepellent may be added to an outlet pool, or separate outlet pools. A chemoattractant and a chemorepellent may be added to one of the outlet pools simultaneously, or consecutively, e.g., after the cells have entered the buffering chamber. The chemoattractant or chemorepellent may be added in the outlet pool or the inlet pool, and both may be added in a single outlet pool or inlet pool. The cells for cell motility screening and/or chemotaxis testing may be added in the inlet pool, or in the outlet pool. More than one chemoattractants and/or chemorepellent may be added to an exemplary microfluidic system, and each chemoattractant and/or chemorepellent may form a gradient along the length of one of the branching channels.

In some embodiments, the method may further comprise laying an oil, preferably mineral oil, on top of the microfluidic device. In some embodiments, the confluency of the cells subject to the cell motility screening and/or chemotaxis testing may be about 25-100%. In some embodiments, the method may further comprise refreshing the cell culture medium. In some embodiments, the chemoattractant and/or chemorepellent may be secreted by a cumulus cell. In some embodiments, the cells subject to the cell motility screening and/or chemotaxis testing may be sperms. In some embodiments, more than one chemoattractants and/or chemorepellents may be added to the outlet pools, wherein each outlet pool may comprise one chemoattractant or chemorepellent.

In some embodiments, the cell motility screening and/or chemotaxis testing may comprise comparing the number of cells moving towards and/or in the branching channels and/or the outlet pools. In some embodiments, the cell motility screening and/or chemotaxis testing may comprise calculating a chemotaxis index (CI), which is the ratio of the number of cells moving towards and/or in the branching channel and/or the outlet pools with the chemoattractant vs. the number of cells moving towards and/or in the branching channel and/or the outlet pools without the chemoattractant, or the ratio of the number of cells moving towards and/or in the branching channel and/or the outlet pools without the chemorepellent vs. the number of cells moving towards and/or in the branching channel and/or the outlet pools with the chemorepellent. In some embodiments, the number of cells is counted at a time point or multiple time points after adding cells subject to the cell motility screening and/or chemotaxis testing to the inlet pool. In some embodiments, the number of cells is counted by video recording. In some embodiments, at least 10, 100, 1000, 10,000 or more cells subject to the cell motility screening and/or chemotaxis testing are added to the inlet pool. In some embodiments, the method may further comprise collecting the cells in the branching channels and/or the outlet pools after the cell motility screening and/or chemotaxis testing.

### D. EXAMPLES

The following examples are offered to illustrate but not to limit the invention.

### Example 1

### Microfluidic Device

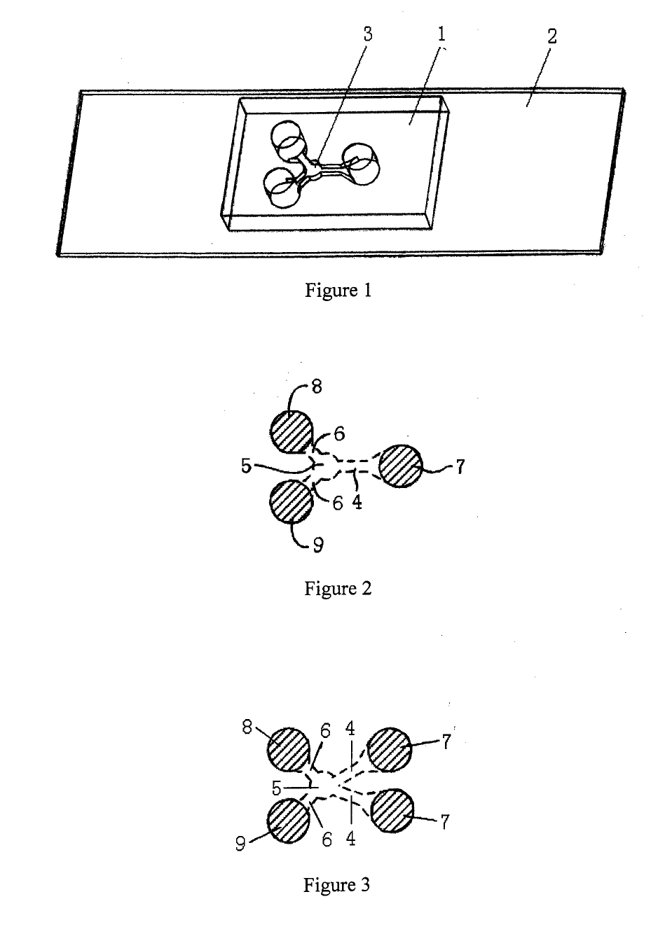

In exemplary embodiments shown in FIGS. 1 and 2, the microfluidic device includes a top layer 1 and a bottom layer 2 and the bottom layer 2 is connected closely to the top layer 1. The top layer 1 contains the microfluidic channel 3 which includes one motility screening channel 4, one buffering chamber 5 and two straight branching channels 6 symmetrically distributed around the buffering chamber 5. The motility screening channel 4 and the straight branching channels 6 are connected by the buffering chamber 5. The inlet pool 7 and two outlet pools 8 and 9 are contained in the top layer, corresponding to the ends of the microfluidic channel 3. The inlet pool 7 is connected to the motility screening channel 4 and the outlet pools 8 and 9 are connected to the straight branching channels 6.

The motility screening channel 4 facilitates cell selection depending on the intrinsic motility of different cells. The motile cells can be collected in the buffering chamber 5, wherein a 2-dimensional chemical gradient can be generated in the buffering chamber 5. The buffering chamber 5 is also used for on-focus counting and observation. The symmetrical branching channels 6 with two outlet pools are used for chemotaxis analysis. Cells secreting chemoattractants are selectively planted in outlet pool 8 or 9 and serve as chemoattractant sources. The microfluidic channel 3 can either be set in the bottom layer 2, or in both the top layer 1 and the bottom layer 2.

In the exemplary embodiment shown in FIG. 3, the number of the motility screening channel 4 can be more than one, whereas the motility screening channel 4 is connected by the buffering chamber 5 in one end and by the inlet pool 7 in the other end.

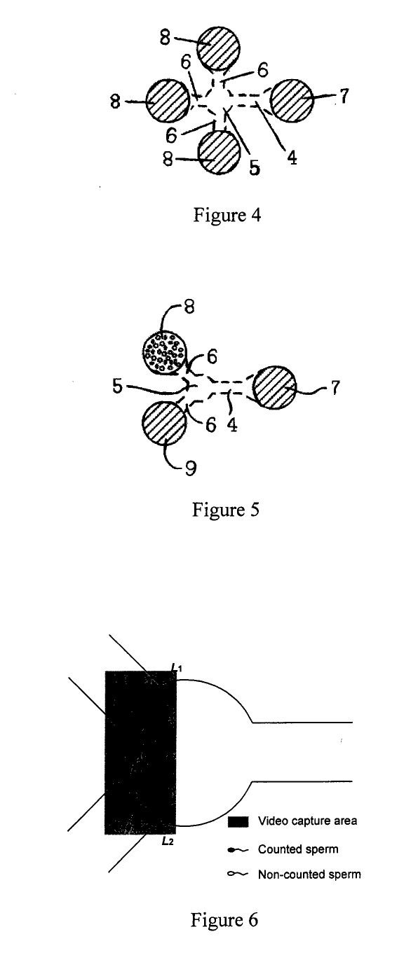

In the exemplary embodiment shown in FIG. 4, the number of the straight branching channel 6 can be more than two, whereas the straight branching channel 6 is distributed symmetrically around the buffering chamber 5.

### Example 2

### Integrated Mouse Sperm Motility Screening and Chemotaxis Assay

In this exemplary embodiment, the top layer 1 is made of PDMS and the bottom layer 2 is made of glass. The microfluidic channel 3 is constructed with standard photolithography and micromolding procedures. SU-8 photoresist is patterned onto a 4 inch silicon wafer to form a master, using printed film as a photomask, and the thickness of SU-8 photoresist will be the final channel height. Liquid PDMS prepolymer solution is mixed by base and curing agent in a proportion of 10:1 and poured onto the master, cured at 72° C. for 1.5 h. The PDMS layer is then peeled off and bonded irreversibly with cover slide by oxygen plasma to form the channel. The specific procedure of plasma bonding is: vacuum the chamber for 1 min, inject oxygen flow at 0.1 MPa for 1 min, turn on the plasma power after the oxygen flow stops for 5 s. After the glow is stable for 15 s, turn the power off and ventilate. Finally, the PDMS and glass slides are taken out and pressed against each other to finish the bonding process.

Procedure

Eight-week-old female ICR mice are super-ovulated by giving an intra-peritoneal (ip) injection of 10 IU of pregnant mare serum gonadotropin 62 h prior to collection, followed by an ip injection of 10 IU of hCG 14 hours prior to collection. Mice are sacrificed by cervical dislocation and the cumulus-oocyte-complexes (COCs) are collected from the oviducts in HTF (human tubal fluid) medium. Three-minute digestion with 3% hyaluronidase at 37° C. is used to separate primary cumulus cells from oocytes. FBS is then added up to a final concentration of 10% to terminate the digestion. The cumulus cells are then spun down at 200×g for 5 min and resuspended with HTF containing 10% FBS.

Using the microfluidic device included the following steps:

1) Before use the entire device is cleaned with ultrasonic washer and sterilized by UV (30 min). Then the device is oxygen plasma treated to improve the hydrophilicity. The specific procedure of oxygen plasma treatment is: vacuum the chamber for 1 min, inject oxygen flow at 0.1 MPa for 1 min, turn on the plasma power after the oxygen flow stops for 5 s. After the glow is stable for 15 s, turn the power off and ventilate.

2) As shown in FIG. 5, the entire microfluidic device is prefilled with HTF. Cumulus cells suspended in HTF are selectively planted in the outlet pool 8 or 9 and cells adhere 5-6 hours later. Cells are usually planted at 60% confluence (approximately 1×10⁴ cells) and are ready for use after 24 hours of culture.

3) Perform chemotaxis assay after cells adhered sufficiently and grew well.

It is important to avoid turbulence of the fluid while planting the cells. To restrict the cumulus cells in outlet pool 8 (or 9), it's necessary to add HTF into outlet pool 9 (or 8) and inlet pool 7 to keep the liquid level in balance.

Mineral oil or other oil is laid on top of the microfluidic device to seal the entire microchannel system. Planted cell amount is determined by the bottom area of the outlet poll 8 or 9. The confluency of the cells is usually in the range of 25-100%. The culture time depends on the cell confluence and growth speed. Usually, the chemotaxis assay is performed after 6-72 hours of culturing.

It is optional to refresh the cell culture medium during the experiment to keep cells in good condition. The concentration of the chemoattractant should be reestimated after medium changing.

To study the effectiveness of the microfluidic device, we set up four groups of experiments:

Experimental group 1: cumulus cells planted in outlet 8 and blank in outlet 9;

Experimental group 2: cumulus cells planted in outlet 9 and blank in outlet 8;

Control group 1: cumulus cells planted in both outlet 8 and outlet 9;

Control group 2: blank in both outlet 8 and outlet 9.

The experimental groups are set to investigate the chemotactic response of sperms. Control group 1 is set to evaluate the symmetry of the growth of cumulus cells. Control group 2 is set to test the symmetry of the microfluidic device. Taken the two control groups into account together, potential bias of the experimental system can be eliminated.

Approximately 25,000 sperms (from male mice, incubated at 37° C. for 30 min for capacitation) are added into the inlet pool 7 of the device. It is necessary to take out 2.5 μl medium right after adding 2.5 μl sperms. After 5-10 min of swimming, sperms start to accumulate in the buffering chamber 5. A 15-min video recording captures sperms heading toward different branching channels. The videos are viewed to count the number of sperms passing through L1 or L2, respectively (FIG. 6). A DP-71 CCD coupled with an inverted microscope is used for video capture (50×).

Results Analysis

Sperm motility screening: For mouse sperms, those with high motility swam forward spontaneously and those with poor motility remained in place. After screening by microchannel, sperm motility (defined as percentage of motile sperm number in the total sperm number) increased from 60% in the inlet pool 7 to 85% in the buffering chamber 5.

Chemotaxis assay: For convenience in evaluating sperm chemotaxis in the current device, we derived a parameter called the chemotaxis index (CI) to assess the characteristics of sperm chemotaxis, which was represented as the ratio of the number of sperm swimming through L1 vs. the number of sperm swimming through L2. Therefore, if sperm chemotaxis was taking place, we would expect the CI>1 for Group 1, <1 for Group 2 and =1 for Group 3 and 4. The result was in accordance with this hypothesis and confirmed the feasibility of the presented invention.

Compared with other methods currently used clinically, the microfluidic device was simple to use and effective in screening. Moreover, centrifugation was avoided which can cause potential damages to sperms. Sperms with chemotactic response can be enriched through the microfluidic device. Since the cumulus cells were utilized as chemoattractant sources, a stable chemoattractant gradient was established in the buffering chamber as well as the straight branching channels. This is superior to other chemotaxis assays for which a stable fluid is difficult to maintain. The continuous gradient contributes to a higher signal-to-noise ratio and mimics the in vivo environment better.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A microfluidic device for cell motility screening and/or chemotaxis testing, which comprises at least one motility screening channel, a buffering chamber and at least two branching channels, wherein the motility screening channel and the branching channels are connected to the buffering chamber.

2. The microfluidic device of claim 1, wherein the branching channels are symmetrically distributed around the buffering chamber.

3. The microfluidic device of claim 1, further comprising an inlet pool and at least two outlet pools, wherein the inlet pool is connected to the motility screening channel and the outlet pools are connected to the branching channels.

4. (canceled)

5. The microfluidic device of claim 1, which comprises a top layer and a bottom layer, wherein the bottom layer is connected to the top layer, the top layer comprises the inlet pool and the outlet pool, and the bottom layer comprises the motility screening channel, the buffering chamber and the branching channels.

6-7. (canceled)

8. The microfluidic device of claim 5, wherein the motility screening channel, the buffering chamber and/or the branching channels are formed between the top layer and the bottom layer.

9-12. (canceled)

13. A microfluidic system comprising a microfluidic device of claim 1 and a chemoattractant, a chemorepellent, or a cell that secrets a chemoattractant or a chemorepellent.

14-15. (canceled)

16. The microfluidic system of claim 13, wherein the chemoattractant or chemorepellent forms a gradient along the length of one of the branching channels.

17. (canceled)

18. The microfluidic system of claim 13, wherein the cell is a cumulus cell.

19. The microfluidic system of claim 18, wherein the cumulus cell is from a human or a mouse.

20. (canceled)

21. A method for cell motility screening and/or chemotaxis testing using a microfluidic device of claim 1, comprising:
a) adding to the microfluidic device a cell culture medium;
b) adding a chemoattractant in one of the outlet pools;
c) adding cells subject to the cell motility screening and/or chemotaxis testing to the inlet pool; and
d) performing the cell motility screening and/or chemotaxis testing.

22-25. (canceled)

26. The method of claim 21, wherein the cells subject to the cell motility screening and/or chemotaxis testing are sperms.

27-28. (canceled)

29. The method of claim 21, wherein the cell motility screening and/or chemotaxis testing comprise comparing the number of cells moving towards and/or in the branching channels and/or the outlet pools.

30. The method of claim 21, wherein the cell motility screening and/or chemotaxis testing comprises calculating a chemotaxis index (CI), which is the ratio of the number of cells moving towards and/or in the branching channels and/or the outlet pools with the chemoattractant vs. the number of cells moving towards and/or in the branching channels and/or the outlet pools without the chemoattractant.

31-34. (canceled)

35. A method for cell motility screening and/or chemotaxis testing using a microfluidic device of claim 1, comprising:
a) adding to the microfluidic device a cell culture medium;
b) adding a chemorepellent in one of the outlet pools;
c) adding cells subject to the cell motility screening and/or chemotaxis testing to the outlet pools; and
d) performing the cell motility screening and/or chemotaxis testing.

36-40. (canceled)

41. The method of claim 35, wherein the cell motility screening and/or chemotaxis testing comprise comparing the number of cells moving towards and/or in the branching channels and/or the outlet pools.

42. The method of claim 35, wherein the cell motility screening and/or chemotaxis testing comprises calculating a CI, which is the ratio of the number of cells moving towards and/or in the branching channels and/or the outlet pools without the chemorepellent vs. the number of cells moving towards and/or in the branching channels and/or the outlet pools with the chemorepellent.

43-47. (canceled)
